(19) United States
(12) Patent Application Publication
(10) Pub. No.: US 2005/0220711 A1
(43) Pub. Date: Oct. 6, 2005

(54) Method and apparatus for stopping and dissolving acoustically active particles in fluid

(75) Inventor: Li-Hai Katz, Haifa (IL)

Correspondence Address: DANIEL J SWIRSKY, PO BOX 2345, BEIT SHEMESH, 99544, IL

(73) Assignee: THERA-SONICS ULTRASOUND TECHNOLOGIES LTD., Zemach (IL)

(21) Appl. No.: 10/520,405

(22) PCT Filed: Jul. 9, 2003
(86) PCT No.: PCT/IL03/00569

§ 371 Date: Jan. 6, 2005

(30) Foreign Application Priority Data
Jul. 9, 2002 (IL) 150656

(51) Int. Cl. A61K 49/00

(52) U.S. Cl. 424/9.5

(57) ABSTRACT

The invention presents a method for selectively slowing the motion of acoustically active particles immersed in a flowing fluid, eventually stopping their motion, holding them in place by pushing them against a surface or against the flow of said flowing fluid, and/or breaking up said acoustically active particles into smaller particles and/or dissolving them. The invention also relates to various systems that utilize this method.

### FIELD OF THE INVENTION

The present invention relates to the handling of acoustically active particles in a fluid. More specifically the present invention relates to a method and apparatus using ultrasound energy to selectively stop, break apart, shrink, and dissolve acoustically active particles immersed in a flowing fluid.

### BACKGROUND OF THE INVENTION

Acoustically active particles, e.g. gas filled bubbles or liquid droplets often are found immersed in a stationary or flowing fluid that is confined within some form of vessel (container, tube etc.). These particles are often undesirable and in many cases are actually harmful, interfering with the flow and/or function of the fluid. In such situations, there is a need to stop their being carried along with the flowing fluid and/or to reduce their size and/or, in some instances, to entirely dissolve them in the surrounding fluid in order to eliminate their potential to do damage.

Bubbles (drops) can be immersed in a fluid in a vessel by two mechanisms:
1. They can be introduced into the fluid from either an outside source (for example: via injection), or as gas released from within a closed container that has been placed in the fluid.
2. They can be formed inside the fluid itself (intra-fluid, intra-vessel) due to pressure changes. Fast intensive injection, turbulent fluid flow (stream), rapid changes in the vessel's dimensions, fluid flow speed, and other causes can all bring about pressure changes, which result in formation of bubbles.

Situations in which it is either necessary or desirable to selectively arrest and dissolve acoustically active particles in a fluid occur, for example, in the food processing industry; fluid transport through pipelines; the flow of fuel, oil, or coolants in machinery or engines; the paint manufacturing industry; etc. The field in which the problem is arguably the most critical and in which a great amount of resources have been invested in attempting to alleviate the problem is the field of medicine. It is from this field that the examples below are drawn in order to describe both the problems created by the presence of the bubbles and the state of the prior art.

Air bubbles or other types of acoustically active particles are introduced into blood vessels during many different forms of invasive procedures. Such procedures include: open heart surgeries, hyperbarics therapy, dialysis treatments (including but not limiting, hemodialysis and hemodiafiltration), minimally invasive stent placement procedures in the cardiac arteries, interventional radiology procedures involving contrast media injection to the cardiovascular system including the cerebral vasculature, and the aorta, X-ray angiographies under fluoroscopy, CT and MRI scans, and during intensive IV (intra-venous) infusions.

Two types of central nervous system (brain) deficits may occur following the above mentioned invasive procedures resulting from the introduction of bubbles into the arterial blood vessels supplying the brain: 1.) focal deficits (stroke) and 2) diffuse cerebral dysfunction, encephalopathy and cognitive damage. Most often these deficits are revealed in the form of subtle mental damage, mild intellectual impairment, confusion or agitation, memory loss, personality changes or depression. When the damage is severe, loss of consciousness, coma, and even death may occur. The parameters which affect the extent of the brain damage due to the bubbles include: their size, the total air volume occupied by the bubbles, and the load (the volume of the bubbles in a given time period). Current techniques for stopping the formation and advancement of the bubbles and air emboli comprise changing the bubble oxygenators to membrane oxygenators at the bypass machines in heart surgery and using barrier filter technology, which is limited to relatively large filter pore sizes mainly ranging from 33 to 40 μm. Pore sizes in the range of cerebral capillaries (7 μm) and red blood cells (8 μm) would improve the filtration of bubbles; but would have a high resistance to flow, would induce more red blood cell trauma, and be a potential source for contamination. Also due to the pressure changes near the filter, large bubbles condense in front of the filter, pass through its pores, exit again and advance towards the brain. Even when using modern bypass machines, studies show, that bubbles are still present at vessels beyond the filter and at the brain [Richard E. Clark, “Microemboli during coronary artery bypass grafting: Genesis and effect on outcome”, Thorac Cardiovasc Surg, 1995; 109:249-258; Borger, Michael A. and J Thorac, “Neuropsychologic impairment after coronary bypass surgery: Effect of gaseous microemboli during perfusionist interventions”, Cardiovasc Surg, 2001; 121:743-749)].

U.S. Pat. No. 5,811,658 [which is based on the article: Karl Q. Schwartz, “The acoustic filter: An ultrasonic blood filter for the heart-lung machine”, J Thorac Cardiovasc Surg 1992; 104: 1647-53] describes a new acoustic filter which can replace or be added to the mechanical filter. According to the method disclosed in this patent, ultrasonic energy is used to divert air bubbles from the main bloodstream to a different chamber where they can be removed. This type of filter can prevent only the bubbles formed at the oxygenator from reaching the blood vessel, but not the following: air bubbles formed at the aorta where the arterial line injects the oxygenated blood at high pressures; emboli formed due to surgical intervention; and air accumulated in the heart, which accounts for most of the air emboli during valve replacement surgeries.

A similar type of device is disclosed in International patent application WO01/41655. The devices described in this publication generate ultrasonic waves that are used to direct the flow of the bubbles in the blood stream directing them to alternate paths or to means to draw them out of the main flow of the fluid into side tubes or by pushing the bubbles to the middle of the tube, where some of them coalesce to form bigger bubbles, and then by sucking them out with a syringe tip placed at the middle of the tube. Neither this publication nor the patent cited above suggests the possibility of stopping bubbles, either from outside sources or those formed intra-vessel, and breaking them up, to accelerate the process of dissolving them, or dissolving them.

Other medical conditions that can be given as examples of situations requiring the utmost care in preventing the introduction of gas bubbles into the blood stream are cerebral and cardiac arterial catheterization and hemodialysis and hemodiafiltration proceedures.

When performing systemic, cerebral and cardiac arterial catheterization it is recommended to extract slowly the contrast media saline from the bottle and inject it slowly to the patient. These procedures cannot always be followed because of the intense and dynamic nature of these interventional procedures. Even if the staff pays careful attention to the formation of bubbles, contrast media must be injected intensely in order to get good imaging of the vessels.

If a patent foramen ovale (PFO) condition is diagnosed in a patient, then the medical staff is encouraged to pay meticulous attention to the formation of air bubbles in intravenous catheters during operations and procedures in intensive care units. PFO is present in one out of four people and for most of them the shunt between the right and left atrium is silent; however, even for a mild condition of PFO, increasing the right atrium pressure (for example by taking deep breath) results in passage of venous blood from the right atrium to the left side. Sources suggest that as little as 2 to 3 ml of air passing through the PFO shunt is enough to cause serious brain damage and stroke.

Another very important situation requiring the utmost care in preventing the introduction of gas bubbles into the blood stream is the problem of chronic air bubbles during hemodialysis and hemodiafiltration. Currently, there are more than 1,000,000 dialysis patients worldwide. Hemodialysis and hemodiafiltration are beneficial treatments in the field of renal replacement therapy for patients with end-stage renal disease. A dialysis patient undergoes more than 150 dialysis treatments, each lasting an average of 3 hours, yearly. During these treatments microbubbles enter the patient's blood circulatory system and cause chronic microemolization in the pulmonary vasculature, which leads to many different types of pulmonary side-effect damage such as pulmonary fibrosis and calcification. In patients with a right-to-left shunt (PFO), paradoxical air emboli can occur and microbubbles may reach the cerebral vasculature resulting in slowly evolving cognitive deficits, which are common in patients on long-term hemodialysis [Yu A. S. and Levy E., “Paradoxical cerebal air embolism from a hemodialysis catheter” Am J Kidney Dis 1997; 29: 453-455; Briefel G. R., Regan F., and Petronis J. D., “Cerebral embolism after mechanical thrombolysis of a clotted hemodialysis access”, Am J Kidney Dis 1999; 34: 341-343].

From the above discussion, it is clear that, especially in cases of PFO, there is an urgent need for a method and apparatus that is able top effectively prevent the introduction of air bubbles into the bloodstream during clinical procedures.

Drug therapy for cancer treatment is another example, also from the field of medicine, of a situation in which it would be desirable to have an efficient method for stopping the flow and dissolving microparticles in flowing fluids.

Cancer is the second largest killer in the world. One in three Americans will eventually develop cancer. These patients are usually treated with surgery, drug therapy, and radiation therapy with many patients given a combination of therapies. Treatment with anticancer drugs may be given intravenously (injected into a vein) or by mouth. The drug travels through the bloodstream in order to reach cancer cells located anywhere in the body. Chemotherapy can be used as the main treatment for the primary cancer or to or in cases where the cancer has spread and metastasized outside of the organ at the time it is diagnosed, or spreads after initial treatments. Neoadjuvant chemotherapy often shrinks the cancer so that surgery can remove cancers that would otherwise be too large for complete surgical removal. Chemotherapy is given in cycles, with each period of treatment followed by a recovery period. The total course of chemotherapy lasts three to six months depending on the regimens used. People having chemotherapy sometimes become discouraged about the length of time their treatment is taking or by the harmful side effects, including fatigue, hair loss, serious heart conditions, nausea and vomiting, loss of appetite, mouth sores, a higher risk of infection caused by a destruction of white blood cells, bruising or bleeding after minor cuts and shortness of breath from which they suffer.

Despite the advances in cancer treatment, there are many areas where the need for effective chemotherapeutic agents remains significantly unmet: advanced prostate cancer, uterus cancer, liver and renal cancer, colon cancer, lung cancer, brain and breast cancer. A treatment for certain types of cancer is hormonal manipulation, which is a non-curative approach. Many patients undergo radiation and chemotherapy treatments. In forty five percent (45%) of newly diagnosed cancer patients and in ninety percent (90%) of patients receiving chemotherapy, cancers are resistant, to varying degrees, to the chemotherapy.

In order to solve or at least lessen the effect of some of the above problems, great efforts are being made to develop site specific drugs, which allow more precise targeted drug delivery to the tumor site. In this technique, chemotherapy drugs are encapsulated in lipid (or other substances) microspheres and can be coated with antigens to be more specific to the cancer cells receptors. The location of the microspheres in the blood stream can be monitored via an ultrasound device and a triggered explosion of the microsphere is possible once the chemotherapy has been absorbed by phagocytes within the tumor.

In order to increase the effectiveness and accuracy of this method of treatment, it would be very useful to have an effective method of slowing, stopping, and accumulating the encapsulated drug at the target site, and rapidly dissolving the outer shell both in the intra and extra vascular regions, therefore releasing the encapsulated drug. In this way the drugs would undergo less systemic cycles and have greater bioavailability in the targeted area. This would result in fewer side effects, and increased intake possibility by the targeted cells. A special catheter can be used in order to release the drugs into the arteries for supplying the targeted site and allowing even more accurate drug delivery.

International patent application WO 02/058530, shows the use of devices in which drug particles are accelerated and then shot on to the surface of the skin. In some embodiments the device is adapted to penetrate the skin before releasing the drug. The disadvantages of this invention are the need to penetrate healthy tissue with the device in order to reach deep sites and the particles acceleration process which is carried out inside the device. As opposed to the method disclosed in this publication, allowing the drugs to taxi independently via the body's vascular system and/or tumor vascular system and accelerating the particles to the vessel walls without penetrating the skin surface with the device would be a significantly improved approach to the problem of drug delivery, both in concept and technology.

It is therefore a purpose of the present invention to provide a method for selectively stopping and/or shrinking and/or dissolving acoustically active particles immersed in a flowing fluid.

It is another purpose of the present invention to provide apparatus for selectively stopping and/or shrinking and/or dissolving acoustically active particles immersed in a flowing fluid.

It is a further purpose of the present invention to provide a method of slowing, stopping, and accumulating an encapsulated material immersed in a flowing fluid at a target site, thus enabling efficient uptake of encapsulated material into the tumor cell.

Further purposes and advantages of this invention will appear as the description proceeds.

### SUMMARY OF THE INVENTION

In a first aspect, the present invention is directed towards a method for selectively slowing the motion of acoustically active particles immersed in a flowing fluid, eventually stopping their motion, holding them in place by pushing them against a surface or against the flow of the flowing fluid, and/or breaking them up into smaller particles and/or dissolving them. the method comprises the following steps:
(a) exposing said acoustically active particles suspended in said fluid to ultrasonic waves propagating through said fluid;
(b) pushing said particles in the direction of propagation of the ultrasonic waves by means of the acoustic radiation force exerted by the waves;
(c) slowing and/or stopping the motion of the acoustically active particles as they enter a friction layer near a surface or surfaces; and
(d) providing an acoustic radiation force having a temporal waveform to act on the acoustically active particles, thereby breaking up the ultrasonically active particles into particles having smaller size and/or causing the particles to dissolve in the fluid.

The acoustic radiation forces for pushing and for breaking up the particles can be produced by either the same or separate sources and can be applied as a superimposition of acoustic radiation forces having two or more frequencies and or waveforms. The waveforms can be either continuous or pulsating.

The method of the invention can comprise the additional steps of:
(i) after step (a), aiming the ultrasonic waves towards the surface of a wall of the vessel containing the fluid or a surface placed in their path;
(ii) after step (b), reducing the speed of the acoustically active particles, which is equal to that of the fluid surrounding them as they are progressively pushed into regions of the fluid closer to the surface; and
(iii) after step (c), pushing the acoustically active particles against the surface by means of the force exerted by the acoustic radiation, thus creating frictional forces between the surface and the acoustically active particles which prevent the movement of the particles and pulsating compressional forces that cause the acoustically active particles to dissolve in the fluid.

In another embodiment of the method of the invention, the acoustic radiation force for pushing and the acoustic radiation force for breaking up are aimed in a direction opposite to the direction of flow of the fluid and along the axis of the vessel through which the fluid flows. The acoustic radiation force for pushing and the acoustic radiation force for breaking up can be focused.

According to the method of the invention, the acoustic radiation force for pushing and/or the acoustic radiation force can be generated upon detection of the acoustically active particles by one or more detectors. The detector can be an ultrasonic detector or an electro-optic detector. The detection can be made by detecting ultrasonic energy emitted by an ultrasonic transducer, refracted by the particles, and detected by either the emitting transducer or another transducer.

The flow of the fluid can be either through a vessel that is open to or surrounded by an object and hidden from view. Ultrasonic detectors, which detect the flow of fluid through the vessel, can be used to aid in determining the orientation of the vessel. The vessel can be located within a human body and can be a blood vessel including a carotid artery.

The surface can comprise one or a plurality of membranes upon which large acoustically active particles break apart into smaller particles that pass through the openings in the membranes upon impact. The size of the pores in the membranes can be between 0.1 μm to 1 mm. The membranes together with the ultrasonic propagating field acting on the acoustically active particles act as a semi-permeable membrane which permits particles to leave the fluid flow through the pores of the membranes and prevents the particles from reentering the flow. An array of open cells can be provided on the side of the membrane surface opposite to the flow of the acoustically active particles and wherein after broken apart particles pass through the pores, they enter the cells thus preventing them from recombining to form particles whose dimensions exceed that of the cells. The pressure exerted on acoustically active particles larger than the pore size of the membrane causes them to deform without breaking apart upon impact with the membrane and slip through the pores, regaining their original shape after slipping through the membrane. In one embodiment, the dimensions of the pores of each succeeding membrane in a plurality of membranes become smaller in the direction of the fluid flow. The surface comprise an array of cells arranged in a honeycomb pattern.

In a preferred embodiment of the invention, the acoustically active particles comprise an encapsulated material, which can be a drug.

In another aspect the present invention is directed towards an ultrasonic system for selectively slowing the motion of acoustically active particles immersed in a flowing fluid, eventually stopping their motion, holding them in place by pushing them against a surface or against the flow of the flowing fluid, and breaking up the acoustically active particles into smaller particles and/or dissolving them. The apparatus comprises:
(a) a fluid flow path through a vessel;
(b) acoustically active gaseous or fluid particles immersed in the flowing fluid;
(c) a surface which creates a friction layer to the fluid that flows adjacent to it, and can be partially or fully submerged in the fluid, or may consist of a wall of the vessel or a type of membrane;
(d) Transducing means acoustically connected to the vessel or submerged in it.

In the system of the invention:
the transducing means delivers acoustic energy having sufficient power to accelerate the acoustically active particles towards the surface where their motion relative to the flowing fluid ceases and to cause breaking apart of the acoustically active particles on the surface;
the acoustic energy being modulated at the optimal deformation frequency of the acoustically active particles, thereby causing safe and selective breakage of the particles into smaller particles which naturally dissolve faster than large particles; and
the acoustic energy being superimposed by harmonic frequencies thereby achieving a negative rectified diffusion of substance from inside the particle to the fluid, or at least lowering the rectified diffusion particles, thus reducing the risk of jet streams and cavitations.

In a preferred embodiment of the system of the invention, the surface is a layer of the flowing fluid and the acoustic energy is directed opposite to the direction of flow. The acoustic energy can be focused and the fluid can be flowing in a tube.

The transducing means can comprise an ultrasound head comprising one or more ultrasound transducers. In some embodiments the number of ultrasound transducers is at least three and two of the transducers are used to detect the presence of acoustically active particles and to influence the operation of the remainder of the transducers. In a preferred embodiment, the transducing means are comprised of a disc shaped main transducer surrounded by an outer ring shaped transducer, the outer transducer being driven in an anti-phase manner to the main transducer. The acoustic energy can be either focused or not focused.

One embodiment of the system of the invention comprises means for providing ultrasonic energy for selectively stopping, breaking apart, shrinking, and dissolving acoustically active particles immersed in blood flowing in the carotid arteries. This system can further comprise a disposable pillow, two ultrasonic heads one located on each carotid artery, and two ultrasonic heads each comprising at least two ultrasonic bubble detectors for detect acoustically active particles and/or fluid flow and at least one ultrasonic transducer to provide the ultrasonic energy.

The surface in the system can be a membrane or have a honeycomb structure to aid in breaking apart and/or holding the acoustically active particles. The membrane acting together with the acoustic energy acts as a semi-permeable membrane, which acts to remove acoustically active particles from the flowing fluid in which they are immersed.

In a preferred embodiment of the system of the invention, the vessel through which the fluid flows is an arterial line of a cardiopulmonary machine, contrast media catheter, or a dialysis machine or a high-flow venous line.

In a preferred embodiment of the system of the invention, the acoustically active particles comprise encapsulated material. The acoustically active particles are delivered to a selected location in a vessel by the flowing fluid, concentrated at the location within the vessel and the encapsulated material is released at the location by shrinking and/or breaking apart and/or dissolving the particles. The acoustically active particles can be introduced into the flowing fluid by use of a specially designed balloon catheter. The encapsulated material can be a drug and the vessel can be part of the vascular system of a human or animal body.

All the above and other characteristics and advantages of the invention will be further understood through the following illustrative and non-limitative description of preferred embodiments thereof, with reference to the appended drawings.

### BRIEF DESCRIPTION OF THE DRAWING

FIG. 1A schematically shows the velocity profile of a fluid flowing in a cylindrical tube;

FIG. 1B schematically shows the velocity curve of a fluid flowing in the vicinity of an arbitrarily shaped surface;

FIG. 9A schematically shows the arrangement of ultrasound radiation, surface, and particle immersed in a fluid that is called for to carry out the method of the invention;

FIG. 2B schematically shows the forces on the particle and the path of its motion as a result of those forces;

FIGS. 3A to 3C schematically show the breaking up of a bubble by abrupt pressure changes;

FIGS. 4A and 4B are photographs showing the before and after state (respectively) of an air bubble held against a bottle wall which was struck hit several times by a plastic pen;

FIG. 5 shows schematically the principle waveform;

FIG. 6 show schematically a preferred embodiment of the ultrasonic head of an apparatus for stopping and dissolving air bubbles in the common carotid arteries;

FIG. 7 is a diagram showing one embodiment of the communication connections between the three transducers of the ultrasound head shown in FIG. 6;

FIG. 8 schematically shows the effect of the ultrasound fields produced by the transducers of the ultrasonic head shown in FIG. 6;

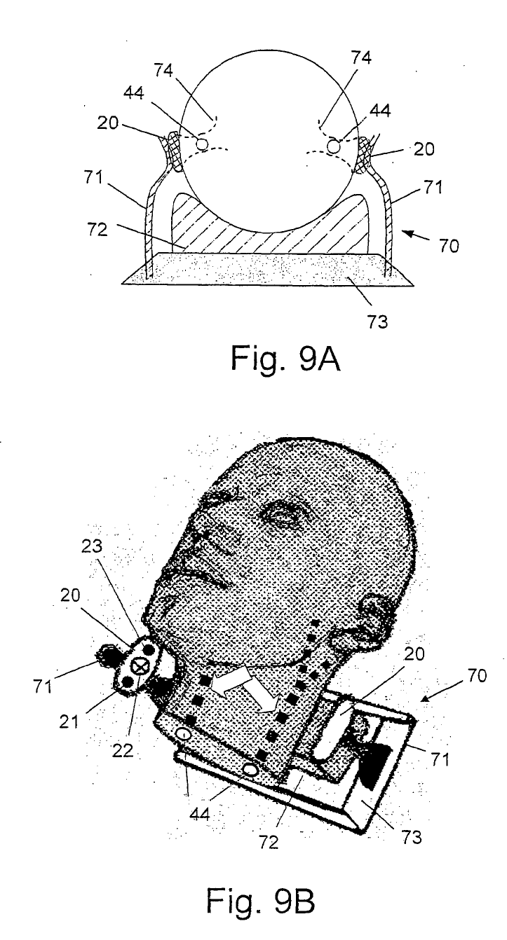

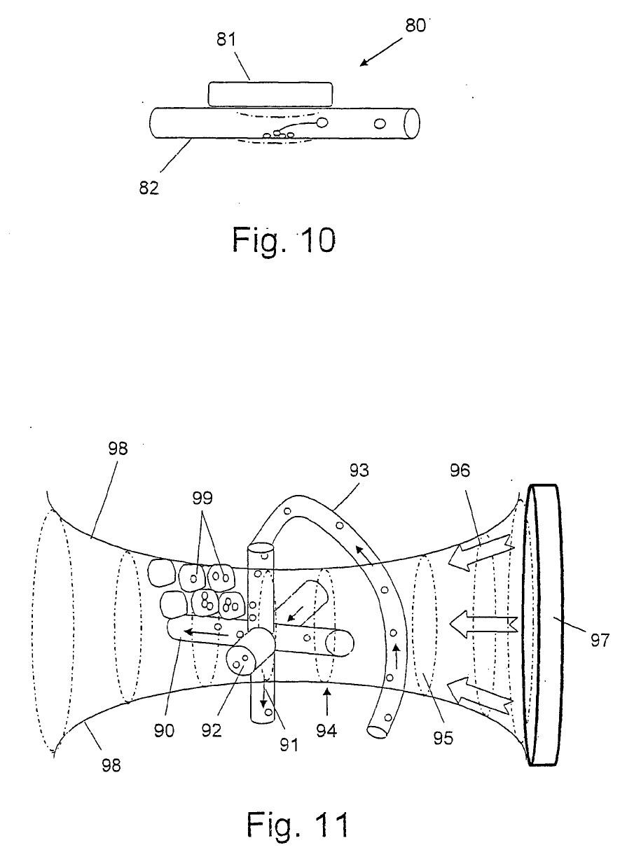

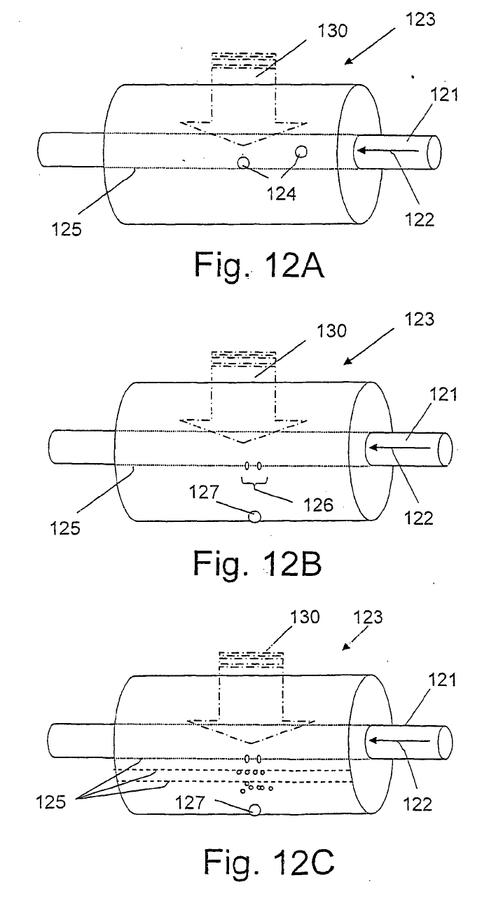

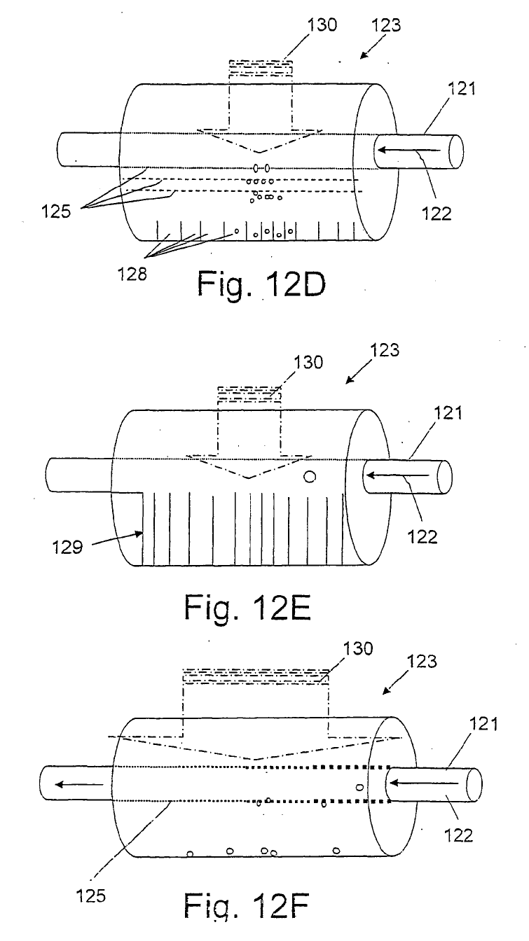

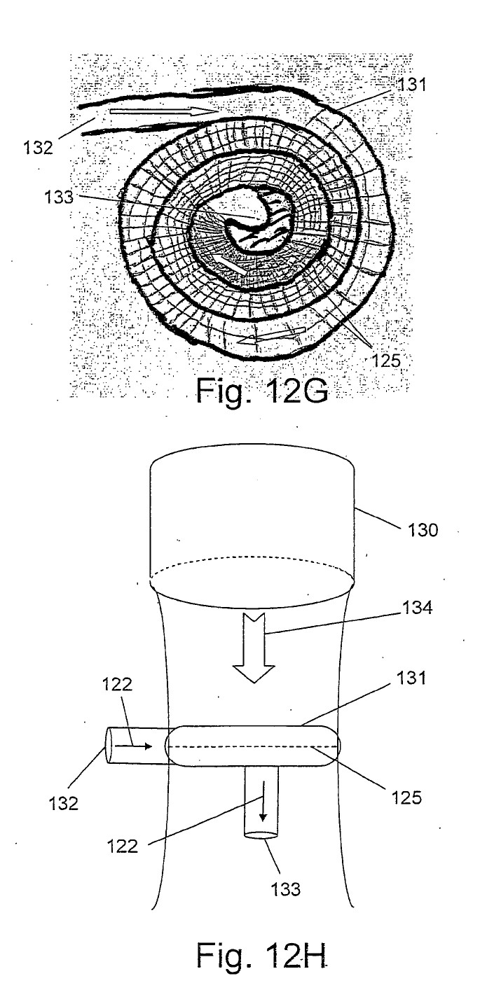

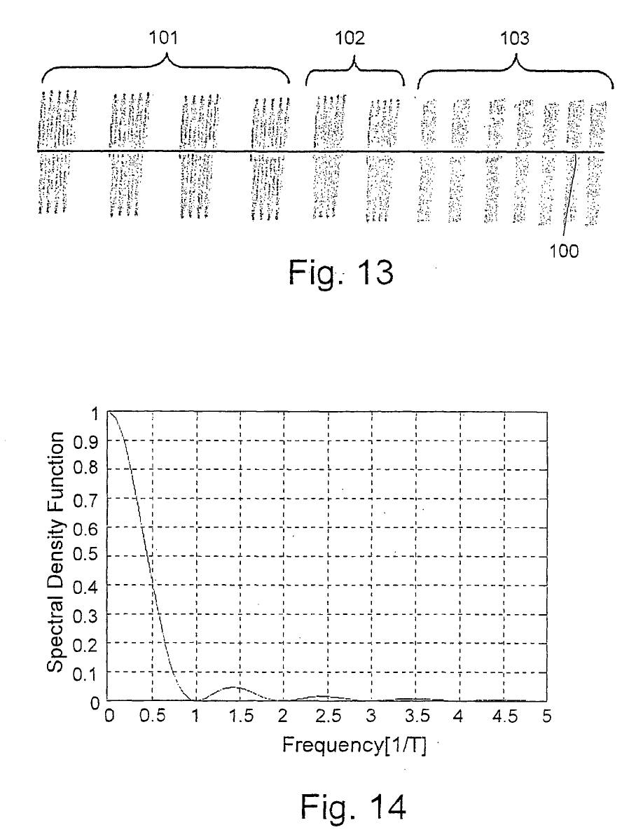

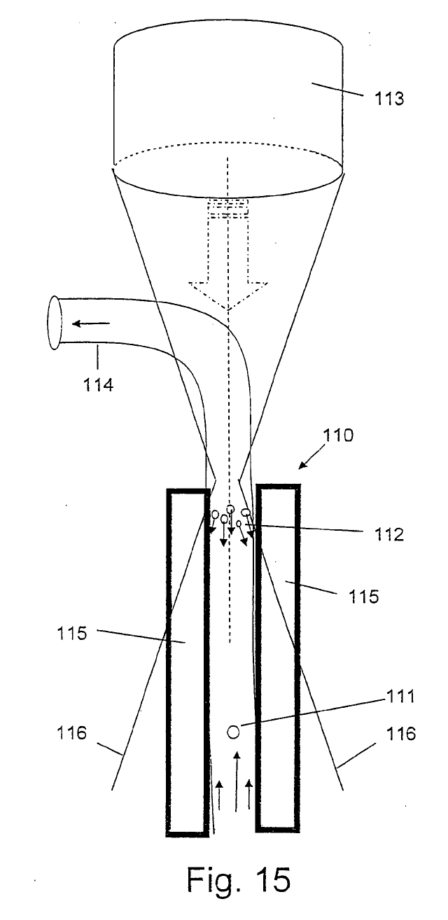

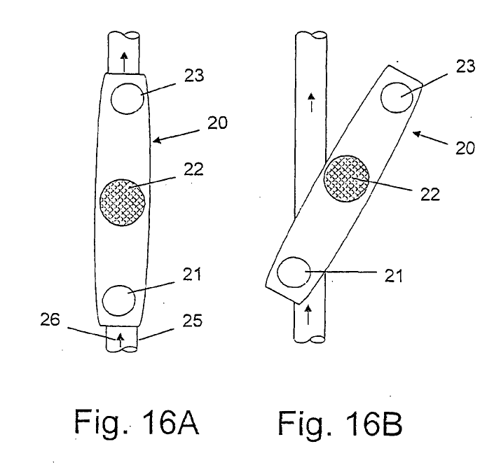

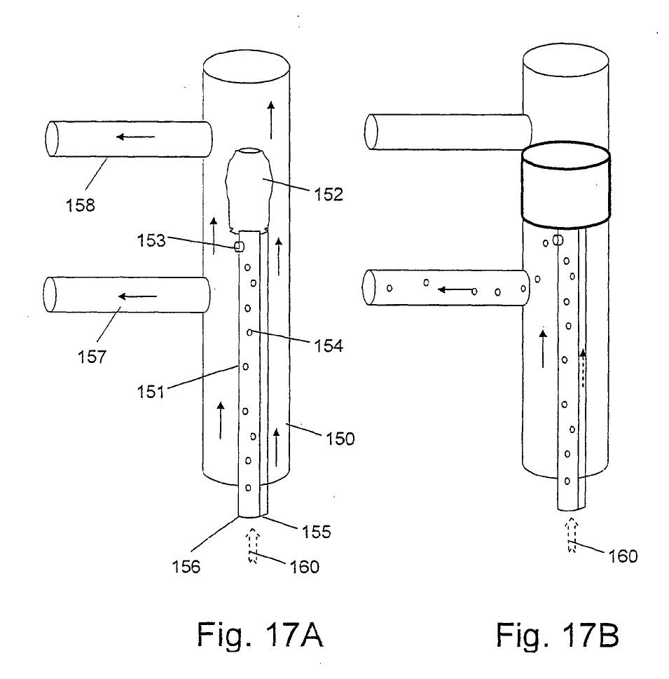

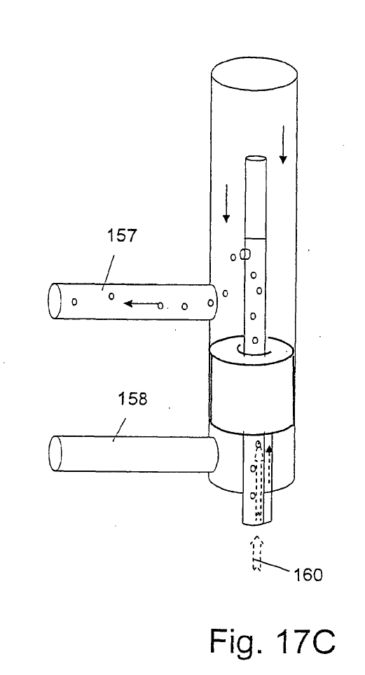

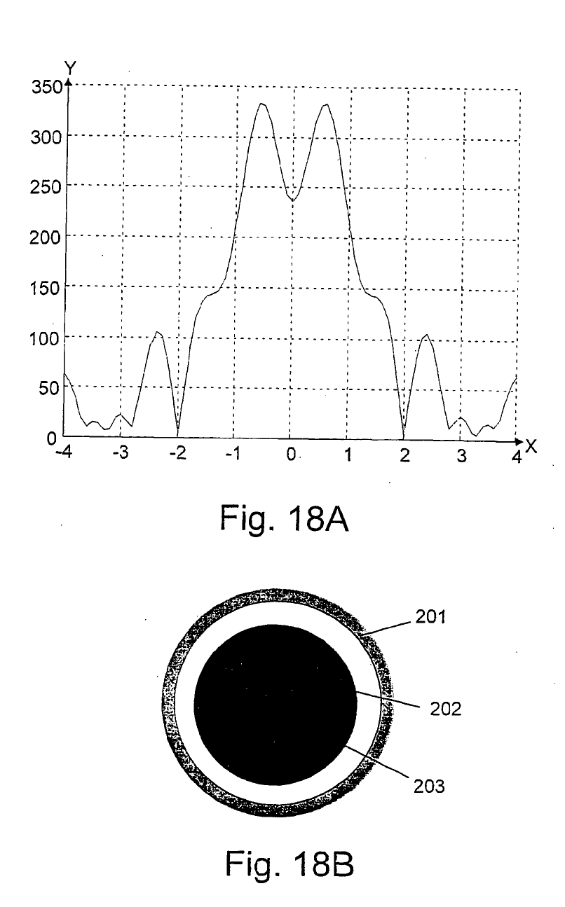

FIGS. 9A and 9B show schematic cross-sectional and perspective views respectively of a preferred embodiment of the device of the invention;

FIG. 10 schematically shows a preferred embodiment of the invention used as an in-line device;

FIG. 11 schematically shows the apparatus used for selectively slowing down, stopping, arresting, accumulating, dissolving the shell, and releasing the material encapsulated within acoustically active particles immersed in a flowing fluid;

FIGS. 12A to 12H schematically show another preferred embodiment of the invention, which comprises a membrane to aid in breaking up and/or holding the bubbles;

FIG. 13 schematically show the ultrasonic wave form used to cause bubbles to oscillate;

FIG. 14 is a graph showing a simulation of the spectral decomposition of a single ultrasonic pulse;

FIG. 15 schematically shows a preferred embodiment of an in-line device;

FIGS. 16A and 16B schematically show how the “Doppler” transducers are used to align the ultrasound head shown in FIG. 6 with the fluid flow direction;

FIGS. 17A to 17C show a non-limiting preferred embodiment of the catheter used to introduce the drugs into the bloodstream; and

FIGS. 18A and 18B schematically show respectively an intensity graph and a simple scheme of the transducer head embodiments of an ultrasonic head that produces a field that confines acoustically active particles to the region of highest ultrasonic pressure.

### DETAILED DESCRIPTION OF PREFERRED EMBODIMENTS

In this application the following terms are used in the following fashion:
The terms “ultrasound”, “ultrasound field”, “ultrasound field of waves”, “ultrasound waves”, “ultrasonic field”, “ultrasonic field of waves”, “ultrasonic waves”, etc. are used interchangeably.
The term “acoustic radiation force” refers to the force exerted on acoustically active particles immersed in a fluid when exposed to a field of ultrasound waves.
The terms “bubble”, “acoustically active particle”, and “particle” are used in a generic sense to include bubbles of all sizes from “microbubbles” (diameters on the order of micrometers) to “macrobubbles” (visible to the unaided eye). These terms also are used to mean “droplet” or “drop”, which, as far as the present invention are concerned, are equivalent to bubble and also such terms as “microspheres”, fluids immersed in other fluids, etc. In general, the term “acoustically active particle” applies to all cases in which one or more fluids are immersed in another fluid that is in a more dispersed phase. Irregardless of whether the dispersed phase is a liquid or a gas, the “acoustically active particle” is the fluid that is immersed in it. The term “bubble” generally refers to an immersed gas, but it can also be relevant when describing a fluid immersed in another fluid. All of these terms can be used interchangeably herein, unless the context prevents this.
The terms “break apart”, “split”, and “shatter”, and similar terms are used interchangeably to refer to the breaking up of a bubble into two or more smaller ones. The term “dissolve” is used to refer to the total break apart of a bubble into individual molecules and their dispersion in the surrounding fluid. The term “shrinking” refers to reducing the size of a bubble according to the method of the invention. The term “neutralize” which refers to shrinking and/or breaking and/or dissolving a bubble till the stage that it is either dissolved or small enough not to interfere with the function of the fluid.
The term “arrest” is used interchangeably with the term “stop” herein, but it further means holding the object in place at the location at which the object's motion was stopped.
Although the fluid is generally referred to herein as “flowing”, it should be understood that the method of the invention can also be used in a static situation, which can be considered to be a special case in which the flow velocity is zero.
The term “transducer” (e.g. piezo-electric element, piezo-ceramics element) is to be understood to also refer to transducer arrays compromised of several transducers, each of which can be excited with a different waveform generator.
The term “pulsating” is used to describe both full on-off modulation of the transducer amplitude and partial modulation, in which case the carrier frequency is modulated with a lower modulating frequency.
The term “membrane” is used to refer to types of surfaces which can be described as “meshes”, “cells”, “netlike”, etc, and all terms are used interchangeably.

As will be described in full hereinbelow, the method of the invention consists of a number of steps which for purposes of convenience in the description of the method and the theoretical background can be divided into two groups, which roughly define two stages in the method. In the first stage acoustic radiation force is applied to slow, stop, and arrest acoustically active particles immersed in a flowing fluid. In the second stage the arrested particles are shrunken or neutralized by either the same acoustic radiation force or one with a different strength or temporal waveform. This division into stages is artificial only and the two stages can be incorporated into a single process and be applied simultaneously.

In order to describe, in a clear and relatively simple way, the method of the present invention for using ultrasonic energy to slow and/or arrest and/or dissolve acoustically active particles, a typical representative situation will now be described. The situation described is that in which a bubble immersed in a fluid that is flowing through a straight round tube enters a region in which ultrasonic waves are propagated in a direction perpendicular to that of the flow direction. The bubble will be pushed by the ultrasonic field in the direction of the wall and will slow down until its motion is stopped and it is held in place against the tube wall. The following theoretical explanation together with the specific examples described hereinbelow will allow skilled persons to intuitively apply the principles of the invention to any situation involving the need to remove acoustically active particles from a flowing fluid in which they are immersed. Using the principles described herein, skilled persons will be able to determine the value of the different parameters involved, in order to achieve optimal results for any given situation. Such a determination will require no more than understanding of the method of the invention and a reasonable amount of trial and error.

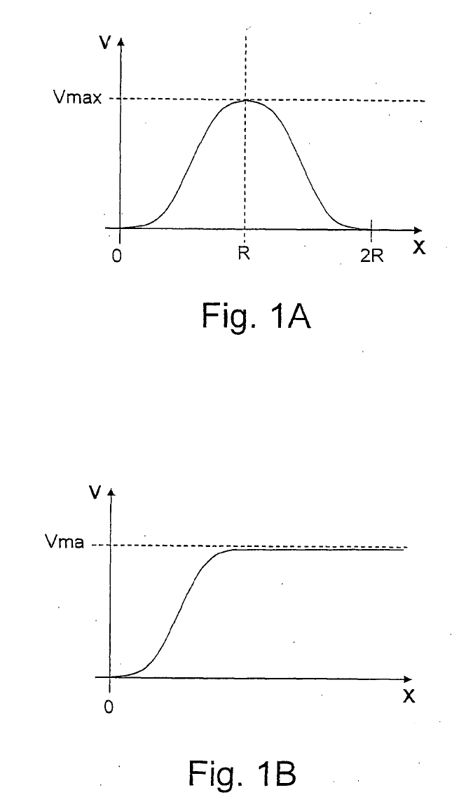

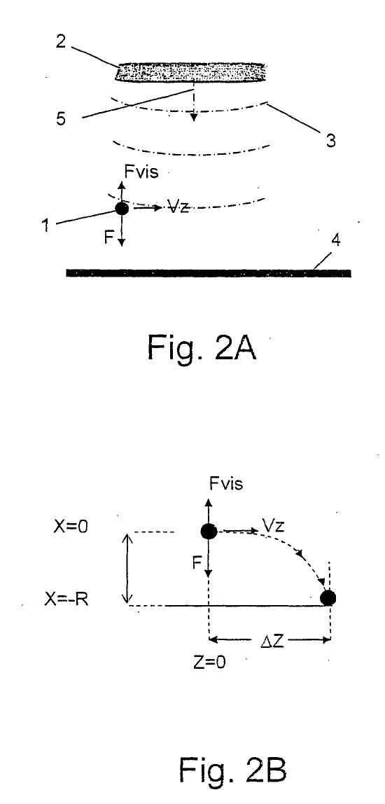

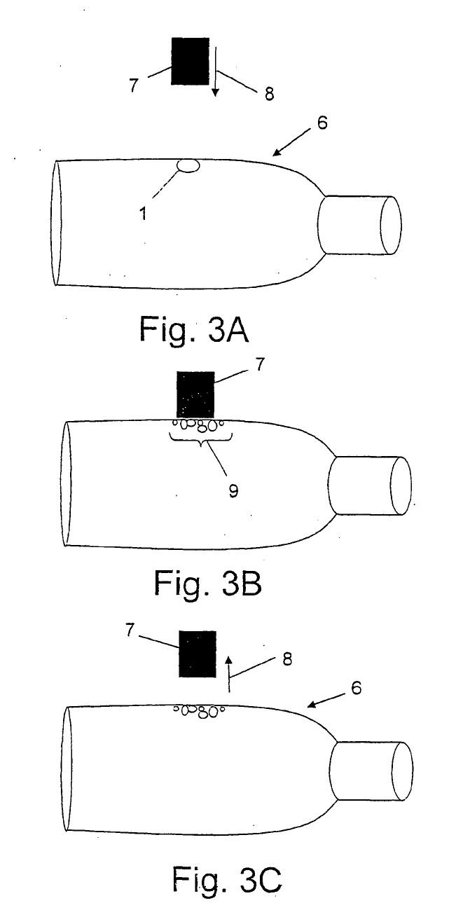

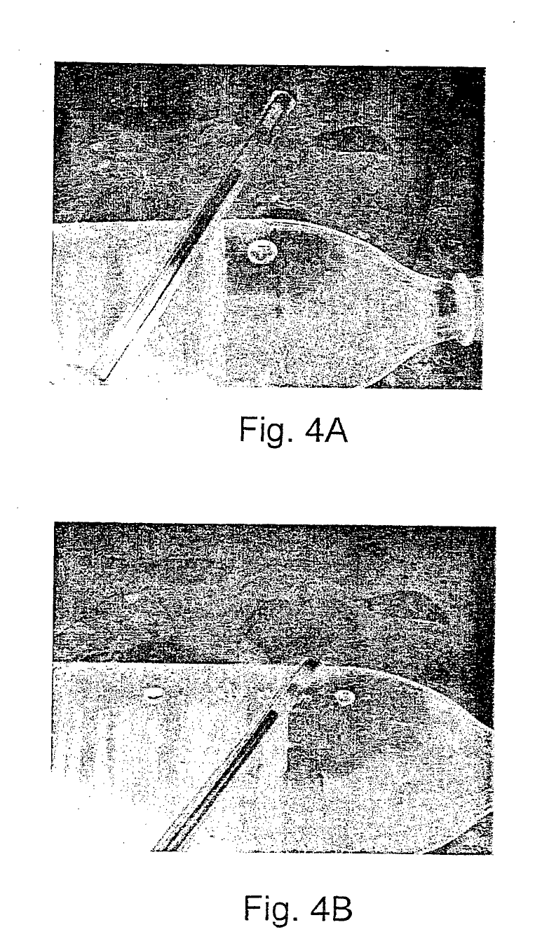

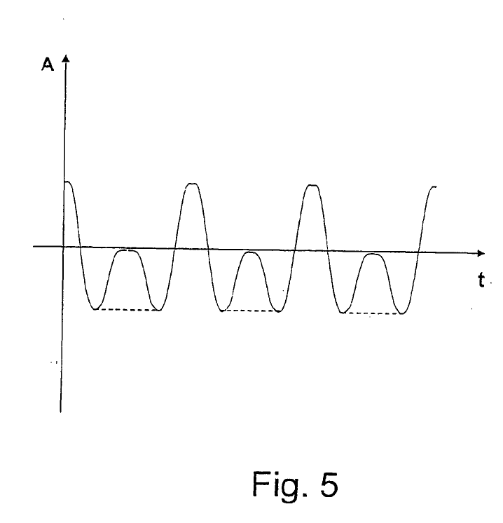

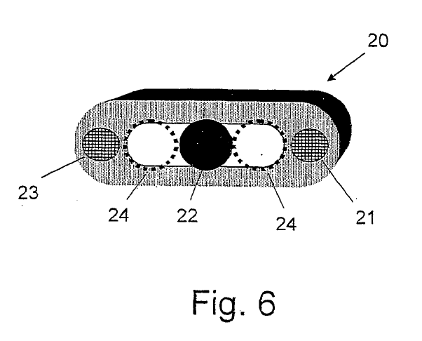

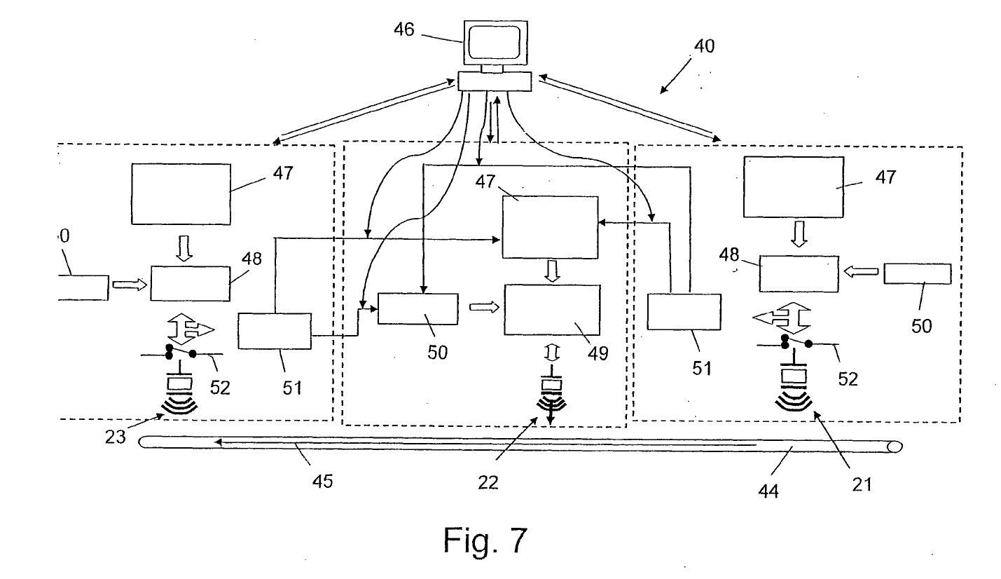

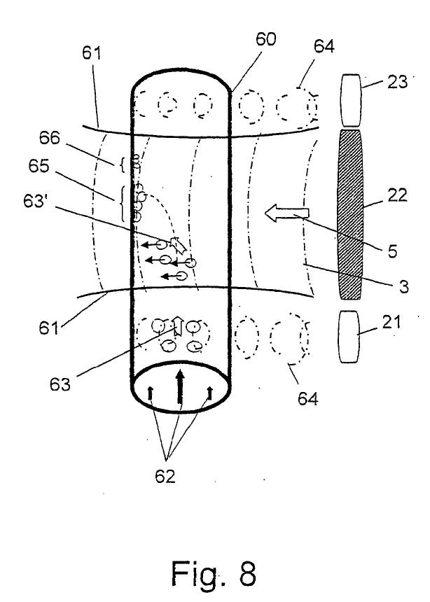

The method of the invention is based on the proper application of several known phenomenon. Firstly it is known that the velocity profile of fluid flowing in a vessel under non-turbulent flow conditions has a parabolic shape. The magnitude of the velocity has its maximum value at the center of the vessel and gradually approaches zero at the vessel wall. This shape velocity profile occurs because of the presence of increased frictional forces near the wall surface. In FIG. 1A is schematically shown the velocity profile for a fluid flowing in a cylindrical tube. The curve represents the magnitude of the flow velocity V a distance X from the wall of a cylindrical tube having radius R.

This parabolic shaped velocity profile is true for a fluid flowing in the vicinity of any arbitrarily shaped surface, not necessarily the wall of the vessel, which is in contact with the flowing fluid. The surface can either be stationary with respect to the walls of the vessel or moving at a velocity slower than the flow rate of the fluid. In FIG. 1B is schematically shown the velocity curve for a fluid flowing in the vicinity of an arbitrarily shaped surface. In this figure, V represents the magnitude of the velocity of the fluid relative to that of the surface and X the distance from the surface.

The essence of FIGS. 1A and 1B is that the velocity of a flowing fluid relative to a surface in contact with it gradually decreasing in value as the distance from to the surface is decreased until it reaches zero at the fluid-surface interface.

A second known phenomenon is connected to the motion imparted to acoustically active particles in an ultrasonic field. Acoustically active particles immersed in a fluid that are exposed to ultrasonic waves traveling through the fluid will be pushed in the direction of the ultrasonic field propagation. Because acoustically active particles are substantially different acoustically from their fluid environment, they are most affected by the ultrasonic energy, and selectively pushed by the ultrasonic force while the pushing effect on the rest of the fluid due to the ultrasonic field is negligible. In the case of a flowing fluid, if a component of the ultrasonic field is propagated in a direction essentially perpendicular to the direction of flow, then the acoustic radiation force exerted on the acoustically active particles when they enter the ultrasonic field will push the particles towards the wall of the vessel through which the fluid flows, or towards a surface placed in the path of the ultrasonic waves, and will eventually push the particles against the surface. At the surface the speed of the fluid is zero and therefore the particles, which are assumed to be carried along passively in the fluid, will come to rest.

The final phenomenon on which this stage of the method of the invention is based is that the magnitude of the frictional force between two objects (in the present case: particles, surfaces, particles and surfaces, etc.) is directly related to the magnitude of the force (i.e. the acoustic radiation force) pushing the objects against each other.

The first part of the process of the invention, i.e. selectively slowing the motion of acoustically active particles immersed in a fluid, eventually stopping their motion, and holding them in place by pushing them against a surface, is carried out by the following steps:
(a) exposing acoustically active particles suspended in a fluid (the fluid flow speed can be zero or greater in any direction) to ultrasonic field of waves traveling in the fluid medium;
(b) aiming the ultrasonic waves towards the surface of a wall of the vessel containing the fluid or another surface placed in their path;
(c) pushing the particles in the direction of the ultrasonic field by means of the acoustic radiation force;
(d) reducing the speed of the acoustically active particles, which is equal to that of the fluid surrounding them (assuming no self-propulsion of the particles) as they are progressively pushed into regions of the fluid closer to the surface, or by the application of an appropriately directed acoustic radiation force;
(e) pushing the acoustically active particles against the surface by means of the acoustic radiation force, thus creating forces between the surface and the acoustically active particles which prevent their movement.

Microbubbles are an example of a type of very acoustically active particles. At ultrasound frequencies near the resonance frequency of the bubble, the scattering cross-sectional area increases by several orders of magnitude above the geometric cross section. The larger the scattering cross-section, the more acoustic radiation force will be exerted on the bubble. It is to be noted that only traveling waves produce the needed acoustic force to push suspended particles and bubbles. Standing waves would only cause the particles to collect at the acoustic pressure nodes or maxima.

In the simplest arrangements, a single-element ultrasound transducer may be used to produce ultrasound (i.e. ultrasonic energy). The strength of the acoustic force on an object depends on the ultrasound direction, frequency and signal strength, and the size, mass and acoustic qualities of the object being acted upon.

Objects that are acoustically different from the surrounding medium are affected differently by the ultrasonic energy. For example, in an artery, spherical air-filled microbubbles have radically different acoustic properties and have much lower mass than biconcave fluid-filled (nonresonant) red blood cells or other irregularly shaped fluid-filled cellular blood elements, therefore the bubbles are preferentially affected by the acoustic radiation force. For an in-depth understanding of the affect of ultrasound on tissues and bubbles the book “Ultrasound In Medicine” edited by F. A Duck, A. C. Baker, H. C. Starritt, Institute of Physics Publishing, of the institute of Physics, London, 1998. see especially Part 4 “Ultrasound and Bubbles” should be consulted.

FIG. 2A schematically shows the arrangement of ultrasound radiation, surface, and particle immersed in a fluid that is required in order to carry out the method of the invention. An acoustically active particle 1 (in this case spherically shaped) is suspended in a fluid. Vz is the velocity vector of the fluid and suspended bubble (in the absence of the ultrasonic field). The horizontal line represents a surface 4, e.g. a wall of the vessel containing the fluid. An ultrasound transducer 2 generates acoustic radiation pressure waves 3 in a direction indicated by arrow 5.

In the example shown in FIG. 2A, the ultrasonic radiation force applied to the particle is F and the mass of the particle is m. As a consequence of the viscosity, a frictional force Fᵥᵢₛ is also exerted on the particle in the direction opposite to that of F.

Fᵥᵢₛ is determined from the following equation:
Fᵥᵢₛ=6πrνη (1)
Where:
r=the particle radius;
μ=the particle velocity
η=viscosity coefficient

The equation of motion is:

$$m \frac{ⅆ v}{ⅆ t} = F - {F}_{vis} = F - KV \qquad ( 2 )$$

Where: K=6πrη

In case the particle is a bubble p is the gas density (for air≈1 Kg/m³).

$$m = ρ \cdot \frac{4 π}{3} {r}^{3} \qquad ( 3 )$$

The solution of equation (2) is:

$$\begin{matrix}V ( t ) = \frac{F}{K} [ 1 - {ⅇ}^{\frac{t}{τ}} ] where \text{:} τ = \frac{m}{K} & ( 4 ) \\ Therefore \text{:} τ = \frac{m}{K} = \frac{ρ \cdot \frac{4}{3} π {r}^{3}}{6 π r η} = \frac{2}{9} ρ \frac{{r}^{2}}{η} & ( 5 )\end{matrix}$$

If the particle is in the order of microns (e.g. a microbubble) it can be assumed that the bubble reaches its limiting speed in a negligible time (for example around 40 μsec for a 20 μm diameter air bubble) thereby simplifying the equation.

Therefore:

$$V = \frac{F}{K} \qquad ( 6 )$$

The acoustic radiation pressure (P_{rad}[N/m²]) is calculated from the ultrasonic power per surface unit of area (Wₐᵣₑₐ[W/cm²]), divided by the speed of sound in the medium (c[cm/s]). If the application of the acoustic pressure is applied to biological systems, than taking into account the very effective heat perfusion to a rapidly streaming blood, radiation power/output of 100-200 W/cm² can be applied for a known period of time which allows for transfer and spread of the heat to the surroundings as the fluid (blood) advances in the body's vascular network without causing excessive heating (similar to a radiator effect). The period of time depends on, among other factors, the fluid volume and flow rate and can be determined by applying the Dewy and Sparto “thermal dose equation” [S. Separeto and W. Dewey, “Thermal dose determination in cancer therapy,” Int. J. Radiat. Oncol. Biol. Phys., vol. 10, pp. 787:800, 1984.] and the Pennes “bio-heat transfer equation” [H. H. Pennes, Analysis of tissue and arterial blood temperatures in the resting human forearm,” J. Appl. Phys., vol. 1, pp. 93:122, 1948].

The force upon the particle will be the radiation pressure (P_{rad}) multiplied by the geometric cross section (the surface facing the direction of propagation of the radiation).

In the case of a spherical particle (e.g. microbubble) the acoustic force will be:
F_{rad}=P_{rad}·πr² (7)

The limiting speed of the particle in the direction of the surface is:

$$V = \frac{F}{K} = \frac{{P}_{rad} \cdot π {r}^{2}}{6 π r η} = \frac{{P}_{rad} \cdot r}{6 η} \qquad ( 8 )$$

Therefore the time it takes for the particle to travel a distance R to reach the surface is:

$$t = \frac{6 R \cdot η}{{P}_{rad} \cdot r} \qquad ( 9 )$$

If for simplicity (as in this illustrative example), the particle is immersed in a fluid medium which moves in a direction that is perpendicular to both the radiation force and the surface, and the surface is flat (note that in general neither the radiation force nor the surface have to be perpendicular to the fluid flow direction and the surface does not have to be flat); then the propagation profile of the particle upstream is described by Bernouli's equation. The velocity of the fluid and the particle decelerates as the surface is approached, until complete arrest of the motion particle is achieved as a result of increased friction forces.

FIG. 2B schematically shows the forces on the particle and the path of its motion as a result of those forces. The forces are as described with respect to FIG. 2A. AZ is the distance, in the z (flow) direction, that the bubble moves parallel to the surface, until it reaches the surface. R is the distance of the bubble from the wall before it is acted upon by the ultrasonic force.

The velocity profile is:

$${V}_{z} ( x ) = {V}_{z} ( o ) [ 1 - {( \frac{x}{R} )}^{2} ] = {V}_{z} ( o ) [ 1 - \frac{{V}^{2} {t}^{2}}{{R}^{2}} ] \qquad ( 10 )$$

While approaching the surface the particle travels upstream a distance of:

$$Δ Z = {V}_{z} ( 0 ) {\int }_{0}^{R / V} ( 1 - {( \frac{Vt}{R} )}^{2} ) ⅆ t = {V}_{z} ( 0 ) [ \frac{R}{V} - \frac{1}{3} \frac{R}{V} ] \\ therefore \text{:} Δ Z = \frac{2}{3} {V}_{z} ( 0 ) \cdot \frac{R}{V} \qquad ( 11 )$$

The properties of the surface (biological, inorganic material, etc.), the particles (gas filled, fluids filled, geometry, etc.), and the surroundings (biological, heat doses, flow velocity, etc.) have to be considered when choosing the properties of the ultrasonic wave to be used.

The second stage of the method of the invention, i.e. the breaking up into smaller bubbles and/or dissolving of acoustically active bubbles that are held in place against a surface will now be described. According to the kinetics of the dissolution process for bubbles in a liquid based on Epstein and Plesset equation [Epstein P S, Plesset, M S, “On the Stability of Gas Bubbles in Liquid-Gas Soluions”, J Chem Phys 18:1505-1509, 1950.], gas bubbles naturally shrink as a result of the surrounding pressure. [Alexey Kabalnov, et. al., “Dissolution of Multicomponent Microbubbles in the Bloodstream”, Ultrasound in Med. & Bio., 1998, 24:739-749]. The estimated for the rate of decrease of the particle radius over time is:

$$\frac{ⅆ r}{ⅆ t} = - DL \frac{{\overline{p}}^{*} + 2 σ / r}{{p}_{atm} + 4 σ / 3 r} \{ \frac{1}{r} + \frac{1}{\sqrt{π Dt}} \} \qquad ( 12 )$$

where D is the diffusivity of air in water, L is the partition coefficient of air between water and gas phase, Pₐₜₘ is the atmospheric pressure, \overline{p}* is the excess pressure, which has a contribution from both the systemic blood pressure and the oxygen metabolism. σ is the surface tension, r is the radius of the bubble, and t is time. The smaller the diameter the faster the bubbles dissolve into the medium. For example, bubbles of around 10001 μm take more than 2 months to dissolve in saturated fluid, 100 μm bubbles take around 10 minutes to dissolve, and a 10 μm bubble dissolves in around 6 sec under the same conditions. Therefore by breaking the bubbles into smaller bubbles a more efficient dissolving process is produced.

One of the mechanisms for breaking up the bubbles is to increase the efficiency of the diffusion process is based on the observation that, if forces due to abrupt pressure changes are exerted on the surface of a bubble, then it will deform and split (break) into smaller bubbles.

The breaking up of a bubble by abrupt pressure changes is schematically shown in FIGS. 3A to 3C. In FIG. 3A is shown a gas macrobubble 1 trapped against the flexible wall of a bottle 6. A fingertip 7 is advancing in the direction shown by arrow 8 towards the bottle wall and the bubble. In FIG. 3B is shown the instant that the fingertip hits the bottle wall and FIG. 3C an instant later when the finger is pulled back. The “whiplash” strike exerts shearing forces on the bubble, breaking it to a group 9 of smaller bubbles. The process can be repeated until the size of the bubbles is reduced to a critical value at which point they dissolve completely in the surrounding fluid.

This phenomenon can be easily demonstrated by holding a macrobubble against the flexible wall of, for example, a standard, 1.5 liter bottle containing water. FIGS. 4A and 4B are photographs showing the before and after state (respectively) of an air bubble held against a bottle wall which was struck several times by a plastic pen.

FIG. 14 is a graph showing a simulation of the spectral decomposition of a 1/T sec⁻¹ long ultrasonic pulse. It can be seen that in the delta-function of a single pulse, most of the energy is concentrated at lower frequencies. By narrowing the pulse more energy is transferred to higher frequencies, but still most of the energy remains at the low frequencies. When generating a Chirp function comprising of multiple modulation frequencies around the correct bubble breakup frequency, close to a bubble's natural deformation resonance, more energy is transferred to the selected frequency, which in turn escalates the bubble oscillations, with the use of less energy and therefore less heat. Alternatively, if the resonance frequency is known only the exact modulation frequency is applied.

The principles described hereinabove are applied, according to the method of the invention, by using ultrasonic energy on gas bubbles immersed in a fluid in a vessel to shrink the gas bubbles and eventually to dissolve them. The process is carried out by different mechanisms which are related to the manner in which the acoustic radiation force is applied to the bubble.

Two techniques that are used to cause stimulated shrinking of gas bubbles according to the method of the invention are based on applying the acoustic radiation force having a temporal waveform to the bubble. The temporal waveform causes shrinking of the ultrasonically active particles faster and more effectively then use of a continuous wave. The ultrasonic waveform can be generated by the same ultrasonic transducer used for moving the bubbles to the wall of the vessel, or by a separate acoustic source.

The first shrinking technique relies on application of a pulsating field to alternately compress and release the bubble therefore increasing the efficiency of the diffusion process. The theoretical principles on which this technique is based are discussed in the article entitled “Enhancement of Sonodynamic Tissue Damage Production by Second-Harmonic superimposition: Theoretical Analysis of Its Mechanism” (S. I Umemura, K. I Kawabata, and K. Sasaki, IEEE Transactions on ultrasonics, ferroelectrics and frequency control, vol. 43, no. 6, 1996) in which it is shown that expanding gas bubbles by rectified diffusion using relatively low harmonic ultrasound frequencies (about 0.5 MHz and 1 MHz) and inducing asymmetric oscillation of bubble pressure with relatively sharp valleys and broad peaks, is feasible.

In the present invention relatively high harmonic ultrasound frequencies (for example about 5 Mhz to 10 Mhz) are employed and asymmetric oscillation of bubble pressure is induced with relatively sharp peaks and broad valleys to achieve the opposite effect to that achieved by Umemura, et. al. This waveform is applied in order to accomplish optimal bubble compression and diffusion of the gas from inside the bubble to the surrounding medium safely and without causing cavitations and jet formation that can be harmful to the surface against which the bubble is held. In FIG. 5 is shown schematically the principle waveform. The pattern of acoustic waves (waveform) can be applied during all or part of the described process, i.e. at any time from the beginning of the pushing until the bubble is finally dissolved. Even if negative rectified diffusion of gas inside the bubble to the surrounding medium is not achievable, reducing the rectified diffusion to zero or close to zero allows the use of higher wave field intensities and therefore greater radiation force for the same mechanical index as that of a pure sine wave, therefore reducing the probability of cavitations and jet stream formation. This is most important in clinical settings where regulatory agencies limit the Mechanical Index that can be applied to living tissues and blood components.

The second technique for accelerating the process of dissolving the gas bubbles is the use of ultrasonic pressure, preferably with the assistance of the surface or wall, in order to cause shape deformation and break apart of a large bubble into a number of smaller bubbles which will dissolve more rapidly into the surrounding fluid. By causing the bubble to oscillate at its natural oscillation frequency a relatively weak pulsating (or modulated) pressure can cause the bubble to break apart.

According to the Hinze equation:

$$N = \frac{Td}{σ} \qquad ( 13 )$$

[Hinze. J. O. “Fundamentals of the Hydrodynamic Mechanism of Splitting in Dispersion Processes.” AlChE J. 1, 289-295, 1995] it can be shown that, if T is the stress caused by the ultrasonic pressure, d is the bubble diameter, and σ is the bubble surface tension, the equation results in the value of the dimensionless quantity N. For certain types of drops and bubbles N is related to the Weber number, which helps to define and characterize the breakup mechanism of the bubble. The larger the Weber number, the more significant is the breakup effect. The smaller the diameters of the bubbles, the greater the acoustic force that must be applied in order to achieve breakup Weber numbers. The probability of breaking bubbles having subcritical Weber numbers can be increased by generating the optimal forcing frequency for the bubble, which is the natural oscillation frequency of the bubble. For the simple case of spherical bubble, the natural oscillation frequency is:

$${ω}_{n} = 2 π f = \sqrt{\frac{( n - 1 ) ( n + 1 ) ( n + 2 )}{{ρ}_{c}} \frac{σ}{{a}^{3}}} \qquad ( 14 )$$

Where σ is the surface tension, ρ_{c} the surrounding medium pressure, for spherical mode n=2 and a is the bubble diameter. The smaller the bubble, the higher its oscillation frequency. See article by [F. Risso “The Mechanisms of Deformation and Breakup of Drops and Bubbles” Multi. Sci. Tech. Vol. 12, pp. 1-50, 2000]

As the bubbles are pressed against the surface by the ultrasonic field, asymmetric pressure surrounds the bubbles (on the sides of the bubble in contact with the surface and the fluid). This enhances the oscillations of the bubbles, which result in fragmentation of the larger bubbles into smaller ones. As discussed hereinabove, the smaller the bubble the faster it shrinks diffuses to the surrounding medium. In contrast to the cavitational effect where the oscillations are associated with volume oscillations, oscillations induced by this technique are isovolumic, therefore deformation induced by this technique are nonviolent and subtle. These oscillations do not cause excessive shearing pressure on the surface violent bubble collapse, and jet formation generally associated with volume cavitations (as appose to shape deformation without changing the bubble volume).

An example of the ultrasonic wave form used to cause the bubbles to oscillate is schematically shown in FIG. 13, which is not drawn to scale. The carrier frequency is either modulated fully (on-off) or amplitude modulated (AM) with modulated frequency which is swept repetitively from a low frequency to a high frequency, and again from low to high and/or from high to low through several or all frequencies in the range in a short time period. As a specific, nonlimitative example, the carrier frequency 100 can be 2.2 MHz and the modulation frequency is swept from 10 KHz to 70 KHz in three steps 101, 102, 103.

The ultrasonic field/s generated by the acoustic source or sources, can be focused to a specific volume or point in the medium in order to increase the acoustic radiation forces at that location.

The ultrasonic field/s can be applied in a continuous state, or can be generated on command by a human operator or automatically by use of an electronic device. The ultrasonic field can be generated after detection of the acoustically active particles by a special ultrasound transducer that uses the Doppler principle or any other detection method known to skilled persons.

Skilled persons will know how to determine the optimal values of the ultrasonic field intensities, duty cycles, frequencies, and the number of acoustic sources, their shapes, dimensions, placement and acoustic properties, for a given application and set of environmental parameters, by applying the principles discussed herein.

Except for cases where it is necessary to use low intensity and low frequencies, ultrasonic waves at frequencies much greater than the resonant frequencies of the acoustic active particles can be used in the method of the invention. In general the ultrasonic frequencies used are about 1 MHz and higher, preferably between 2 MHz and 10 MHz. In the particular case of air microbubbles, frequencies in the range of about 1 MHz and higher can be used. These frequencies are chosen to avoid cavitations and jet formation that could damage the fluid or surface.

The methods discussed hereinabove for selectively stopping and dissolving gas in moving fluids will now be applied to the design of several devices in order to illustrate how the invention can be applied in specific situations. The embodiments of the device of the invention described herein below are meant to be illustrative only and not limitative. Although the examples chosen are from the field of medicine, it is again stressed that the method of the invention will be useful in many industrial situations from many different fields.

The sources of the ultrasonic energy (transducers) have to be able to create fields with different magnitudes and wave forms and be able to perform different functions such as detection, determination of particle size, pushing, arresting, and breaking up the bubbles in the different embodiments of the invention described herein. Before describing specific embodiments, some general methods of operation of the transducers will be described in order to give the skilled person enough information to adopt the method of the invention to any possible situation.
1. A method that does not detect the bubbles or measure their sizes (“shooting blind”): A single generator generates a pulsating carrier (main frequency) in a chirp mode as shown in FIG. 13. The waveform shown translates into a modulated ultrasonic radiation field. When different sized bubbles pass through it at the same time, each will oscillate and break apart when the correct pulsating and/or modulated force is exerted upon it. A large bubble passes through several pulse cycles (or regimes) until it breaks into increasingly smaller bubbles. The pulsating ultrasonic force will also push the bubbles towards the vessel wall or surface causing them to be arrested against the surface. In the case of a net or honeycomb cell at or before the surface, as will be explained hereinbelow, the large bubble will break, on impact, into smaller bubbles that are arrested behind the net or inside the cells).
2. Another “blind” method: The generator is caused to pulsate or is modulated at the chirp modulation frequencies given with reference to FIG. 13, while always maintaining a low intensity CW (continues wave) signal in order to arrest the bubble already stuck at the friction layer, further preventing them from moving.
3. Using one or more transducer and generators which deliver different carriers simultaneously, by chirping all the carriers with different modulation frequencies, several different bubble sizes can be handled and broken up at once.
4. Using an ultrasound, electro-optic, or other type of detector, in order to detect incoming bubbles and activating the breaking transducer only when there are bubbles present and/or automatically adjusting the modulation frequency to the bubble size and shape. Another detector can be used downstream to assure that no bubbles have passed the bubble breaking/dissolving transducer.

All the above methods can comprise the superimposition of two or more frequencies in order to further shrink the bubbles, or allow higher ultrasound intensities without causing cavitations.

The purpose of the preferred embodiment of the device of the invention schematically shown in FIGS. 6 to 9B is to stop and dissolve air bubbles in the common carotid arteries. In FIG. 6 is schematically shown a preferred embodiment of the ultrasonic head of the device.

The ultrasound head 20 comprises of two “Doppler” elements 21 and 23 the main ultrasound transducer 22 and expansion slots to allow the attachment of additional transducers 24 if desired in order to give the device better arrest and dissolve capabilities. The length of the head is about 5 cm or shorter, to fit the length of the common carotid artery of an average human. A pediatric version of the invention should be shorter.

The first “Doppler” element 21 of head 20 is an acoustic source (e.g., piezo-electric transducer) capable of detecting blood flow in the carotid artery by analysis of the Doppler effect and distinguishing between blood free of acoustically active particles and the presence of bubbles in the blood. Suitable acoustic sources with the required capabilities are common in the art and are commercially available.

The second transducer (or transducers) 22 is the acoustic source described hereinabove for carrying out the method of the invention, i.e. safely and selectively stopping, breaking apart, and shrinking the bubble. In this case the surface against which the bubbles are arrested is the arterial wall and the pushing and shrinking process is accomplished by modulating the frequency to the optimal breaking frequency, and breaking the bubbles into smaller bubbles that dissolve more rapidly. This process can also be accompanied by the use of superimposed waveform as shown in FIG. 5 to allow the use of higher ultrasonic fields while still avoiding the rectified diffusion process that might cause cavitations. In cases where there is no danger of damage to the vessel wall, the bubbles can be made to hit the vessel wall with sufficient momentum to split them into smaller bubbles upon impact. In either case, after the bubbles reach the wall the acoustic element keeps pulsating, in order to break the bubbles held against the vessel wall into smaller and smaller bubbles as described hereinabove.

The third transducer 23 has the same acoustic properties as the first one. It detects blood flow, and air bubbles (acoustically active particles) in the blood. If bubbles manage to pass the second transducer, the third one detects them and alerts the user, and/or changes the second transducer's acoustic output via a feedback mechanism in order to improve the efficiency of the process.

Air bubbles, suspended in the bloodstream passing through the carotids, are detected by the first transducer and selectively and safely neutralized (stopped, broken up into smaller bubbles, and shrunk by use of a special waveform designed for this purpose) by the second transducer. The third transducer provides confirmation that the bubbles detected by the first one have been neutralized by the second and provides feedback to the second transducer if necessary.

FIGS. 16A and 16B schematically show how the “Doppler” transducers are used to align the ultrasound head shown in FIG. 6 with the fluid flow direction 26, in cases where the vessel 25 through which the fluid flows is hidden from view. The first 21 and third 23 transducers in the ultrasonic head locate the vessel 25 by sensing the fluid flow through it. By comparing intensities of the signals detected by both transducers, the alignment of the long axis of the ultrasonic head with the fluid flow direction can be achieved.

In this preferred embodiment, the inputs of the first and third transducers are connected to the second transducer's output, in order to apply a suitable ultrasound output for pushing the bubbles to the wall within the ultrasound waves field; and, at the same time, to apply the exact waveform required for shrinking the bubbles, according to the parameters of the bloodstream, diameter of the bubbles, bubble volume, etc.

FIG. 7 is a diagram showing a preferred embodiment of the communication connections between the three transducers of the ultrasound head of the preferred embodiment described above. The electronic components 40 are activated by computer (or a microcontroller, chip, etc.) 46 which is controlled by appropriate software or embedded in the hardware. In the figure, tube 44 represents the carotid artery and arrow 45 the direction of blood flow. The first, second and third transducers are respectively designated by numerals 21, 22, and 23. The rest of the components shown are: duty cycle establisher multivibrator circuits 47, amplifiers 48, voltage controlled amplifier 49, waveform generators 50, oscilloscopes/FFTs (Fast Fourier Transform) 51, and switches 52.

FIG. 8 schematically shows the effect of the ultrasound fields produced by the transducers of the ultrasonic head described hereinabove on acoustically active particles 1 (e.g. gas bubbles or liquid drops) immersed in a fluid flowing in a vessel 60 (e.g. a plastic tube or pipe or a carotid artery). The black arrows 62 indicate the velocity vectors of the fluid flowing in the vessel (faster flow speed towards the middle). Bubbles 1 traveling through the vessel in the general direction indicated by white arrow 63 are detected by a “Doppler” acoustic source 21 capable of detecting acoustically active particles in a medium by sending, receiving, and analyzing ultrasound energy 64. The source 21 is also capable of detecting flowing fluid, like blood flowing in the carotid. After the bubbles have been detected by the “Doppler” source, the main acoustic source 22 is activated creating acoustic radiation pressure waves 3. The ultrasound waves propagate in the general direction of the white arrow 5 and the black arches 61 indicate the boundaries of the ultrasonic field generated by transducer 22. The focus can include the vessel and the surrounding, all of the vessel or part of it. The focus site (point or volume) is not limited to a specific shape or size, and is determined by the properties of the acoustic source (or sources) in order to achieve the best stopping and dissolving capabilities for a given set of conditions As the bubbles enter the ultrasonic field, acoustic force is exerted on them in the direction of the field 5, pushing them towards the vessel's wall. At the same time, they are advancing with the flowing fluid. The direction of their motion is shown by arrow 63′. As they approach the wall they are slowed down because of increased friction between the fluid and the wall, until they eventually stop moving and are held against the wall. This situation is indicated in the figure by numeral 65. The bubbles are next broken into smaller faster diffusing bubbles as explained above. Another “Doppler” source 23 monitors the vessel for any remaining bubbles and provides a feedback loop for the system. The feedback loop can be used to change the parameters of the main acoustic source 22 in order to achieve better arrest and shrinking capabilities.

When a bubble is placed in an ultrasonic field, the radiation force pushes it forward. It will however, also move side ways toward areas where the force is minimal. If however, the radiation field is as is shown in FIG. 18A., (where the X axis represents the distance from the transducer's central axis and the Y axis represents the pressure intensity) and the bubble is initially at the center of the field (the inner field), it will move forward only. Such a field is easily produced by a transducer, which is for exampled comprised of a circular transducer to which an outer ring has been added, as in FIG. 18B. The outer ring-shaped transducer 201 is driven in anti-phase to the main disc-shaped transducer 202. When a bubble is trapped inside the inner field 203, it can not escape because the higher pressure at the perimeter diverts it back towards the center.

The method of stopping the bubble by pushing it to the vessel wall or surface and breaking the bubble into smaller bubbles by applying a pulsating frequency can now be carried out as described herein. The shape of the head is not limited to circular and can be, for example elliptical or rectangular. The ultrasonic head can be focused at any distance or not focused and the field produced can be used to trap a single bubble or a group of bubbles at the same time.

FIGS. 9A and 9B show schematic cross-sectional and perspective views respectively of a preferred embodiment of the device of the invention. The device 70 comprises two ultrasonic heads 20 one for each of the two carotid arteries 44, one of which is located on each side of the neck. The dashed lines 74 in FIG. 9A schematically represent the boundary of the ultrasonic field inside the neck The ultrasonic heads 20 are situated on adjustable supports 71 which allow freedom of movement of the ultrasonic heads in all directions to permit easy adjustment and precise alignment of the heads with the arteries. The patient's neck is placed on a specially designed inflatable head and neck pillow 72 (made from foam or sponge, etc.), in order to prevent acute changes of the positioning of his head and neck. The base of the apparatus 73 can contain the electronics for the device, or the electronics can be placed in a separate container. Other instruments (monitor, user interface, etc.) should be placed in the most convenient manner. This embodiment of the invention is a device that can be used for the supply of blood that is free from microbubbles and particles from a heart-lung machine or on the patient's neck during open-heart surgery and other invasive procedures to prevent the harmful microemboli from reaching the brain.

Another preferred embodiment of the invention is an in-line device for stopping and dissolving air bubbles embedded in a fluid flowing through a line, i.e. a tube or pipe. Some examples from the field of medicine of lines with which this embodiment can be used are: arterial lines of a cardiopulmonary machine, contrast media catheters, dialysis machines, and high-flow venous lines. This embodiment is a simplified version of the embodiment described hereinabove. In its basic form, the device comprises only one transducer but, in the preferred versions has a transducer array. The “pushing” acoustic element (e.g. piezo-electric transducer or transducer array) creates an acoustic radiation pressure field as described hereinabove. The element supplying the acoustic energy is turned on and off in a special cycle regime that is determined to accomplish the best arresting and dissolving capabilities for a specific line. The bubbles are pushed towards the vessel wall by the pulsating high frequency ultrasonic energy field, the bubbles may only be stopped at the vessel wall or, since there is no danger of damage to the wall in this case, they can be made to hit the vessel wall with sufficient momentum to split them into smaller bubbles. In either case, after the bubbles reach the wall the acoustic element keeps pulsating, in order to break the bubbles held against the vessel wall into smaller and smaller bubbles as described hereinabove.

FIG. 11 schematically shows a preferred embodiment of the in-line device 80 described in the previous paragraph. A piezo-electric transducer 81 is attached by being clipped, glued, threaded, or by any other suitable means to a hollow tube 82 (the line) containing fluid flowing through it. For this use, a preferred ultrasonic cycling regime consists of an ultrasonic energy pulse for the time it takes a bubble to reach the vessel wall with sufficient momentum to be deformed and to split into smaller bubbles by the shearing forces on the bubble followed by about one cycles of rest. For example, if ultrasonic energy of about 100 W/cm² is applied it takes about 20 msec for a bubble with a radius of 10 μm to reach the wall of a vessel 0.8 cm in diameter; Therefore a cyclic regime comprised of a 20 msec ultrasound pulse followed by 20 msec of rest can be used (1:1 ratio). To increase the efficiency, each pulse can be further modulated at the bubble's deformation frequency. (refer to the equation number 14 above)

Other embodiments of the in-line device can incorporate bubble detectors (ultrasonic, optical etc.), and superimposition mechanisms as described hereinabove and can be focused and adjusted to best fit a given situation. In the case of the medical examples mention above, the apparatus prevents dangerous particles and air bubbles from entering the body's blood circulation system and reaching vital organs in the body, where they can cause ischemia and damage.

FIG. 15 schematically shows a preferred embodiment 110 of the in-line device described in the previous paragraphs. In this embodiment the fluid line 114 and the medium surrounding it preferably has an acoustic impedance close to that of the flowing fluid. The line (tube) is bent so the fluid flows (flow velocity indicated by dark arrows) towards the ultrasonic head 113. The ultrasonic head is focused on the axis of the fluid line, where the flow is the fastest. A bubble 111 flows in the fluid (the bubbles tend to flow at the center of the tube) in a region where it is not affected by the ultrasonic field. The field generated is modulated at the bubbles optimal breakup frequency (by finding the bubble size using a detector, or by generating a predetermined chirp waveform). The transducer generates a force field which functions as a selective bubble barrier. This barrier can serve two functions: to select which size bubbles can pass and which cannot and also to isolate the volume of the fluid in which bubbles are immersed from bubble-free regions. When the bubble 111 approaches the focal region, it is broken up into a group of smaller bubbles 112. These bubbles are pushed backwards, against the flow direction and then advance again, being broken down into even smaller bubbles when the reenter the focal region. The larger the bubble the larger the force exerted on it pushing it backwards. Because the field is focused it tends to spread out after the focus, sending the bubbles back and toward the wall of the tube. The intensity of the ultrasonic waves can be determined to allow bubbles smaller than a certain size to pass the “ultrasonic barrier” or not to allow any size of bubble to pass, i.e. to force all bubbles to dissolve completely into the fluid. In FIG. 15, numeral 115 represents a support for the tube and/or a shield to which absorbs the ultrasonic energy outside of the tube. Curved lines 116 designate the shape of the ultrasonic field.

FIGS. 12A to 12H schematically show another preferred embodiment of the invention. The membrane (e.g. cells, net, mesh) is a type of surface with unique attributes (pores). The membrane acts as a semi-permeable membrane which, together with the ultrasonic propagating field, further enhance the capabilities of the method for arresting, breaking and dissolving acoustically active particles. This embodiment can be used with, for example, blood lines of dialysis and heart-lung machines, high-flow infusion lines, different types of infusion pumps and power injectors. In this embodiment the surface against which the acoustically active particles are stopped has a honeycomb or netlike surface facing the fluid. Acoustically active particles are accelerated towards the membrane by the ultrasound field in order to achieve one or all of the following effects:
breaking bubbles larger than the size of the membrane pores with the bubble fragments then pushed by the ultrasound force through the membrane;
deforming large bubbles and squeezing them through the membrane pores; and
passing bubbles smaller then the membrane's pore size through the membrane surface or shatter them on the grid lines.

The membrane and the ultrasonic field prevent the reentry of particles that have passed through the member from reentering the main fluid flow. In the area between the membrane and the vessel the friction is high and the flow speed is low, therefore less energy is needed to keep the acoustic active particles in position.

The system of the invention has advantages over the prior art mechanical filters for many uses. For example, as mentioned hereinabove, the pore sizes of mechanical filters at heart-lung machine arterial lines is limited in size in order not to compromise the blood particles, therefore many bubbles manage to pass the filter and enter the body. In contrast, the pore size in this preferred embodiment of the invention is not limited since the ultrasonic waves are differential and selectively affect the acoustically active particles, while the remainder of the fluid remains unaffected.

Referring to FIG. 12A, the particles (bubbles) 124 enter the device 123 through the line 121. The fluid flows in the direction indicated by the arrow 122. The bubble initially travels along the axis of the tube until it enters the ultrasonic field generated by transducer 130 at which point it is pushed by the ultrasonic force in the direction of the membrane 125. It is to be noted that the membrane, as is the case with all of the preferred embodiments of this invention, can be designed and engineered by skilled persons to provide maximum effect at minimum cost.

FIG. 12B shows the breakup of the bubble into a group of smaller bubbles 126 as it hits the membrane. Breakup occurs because of the large and abrupt forces exerted on the bubbles as it impacts the grid of the membrane, as explained in further detail hereinabove, most of the energy due to the impact is located at the lower frequencies (see the delta-function of a single pulse in FIG. 14). As can be extrapolated from the equation 14, the larger the bubble's diameter the lower its natural deformation oscillation frequency (and vice versa) and therefore large bubbles more easily break into smaller bubbles. After the bubbles pass through the membrane the small bubbles may naturally merge again to form larger bubbles 127. In this case both the membrane and the ultrasonic field will prevent the bubble from returning to the main field. As explained above, the ultrasonic energy exerts greater force on larger bubbles. Thus, pushing a large bubble to the wall and preventing it from moving can be done with much less ultrasonic power (and heating) using this embodiment than using an embodiment without the membrane. As in the other embodiments described herein, the ultrasonic field can be made to pulsate at optimal deformation frequencies to assist in breaking apart the bubbles.

In FIG. 12C, instead of a single membrane, the bubble passes through several membranes having increasingly smaller openings. By timing the ultrasound pulses the bubbles strike the membranes and split into smaller bubbles (which take less time to dissolve in the surrounding medium), the bubbles which have not dissolved merge again; or, as shown in FIG. 12D, are pushed into small cells 128 where they cannot merge to form a bubble larger than the cell. If the cell size is smaller than the size of the original bubble then the bubbles in the cells will dissolve more quickly than the original bubble.

FIG. 12E shows an embodiment where instead of a membrane, cells 129 (of appropriate shape and dimensions) in a honeycomb pattern (side by side) are used. In cases in which the fluid is blood, the cell walls and membrane can be coated with heparin or other anticoagulant substance. The anti coagulant substance can also be spread on a sponge like materiel in the cells or around the membranes. The outer wall of the cell can be covered with an acoustically matching substance (such as gel), for minimal losses during ultrasonic energy transfer.

In FIG. 12F is shown an embodiment in which the membrane 125 is made with increasingly smaller holes (typically 0.1 μm to 5 cm in clinical scenarios) with the direction of the flow indicated by the black arrows 122. As described above the acoustically active particles is accelerated toward the membrane by acoustic force. As discussed above, larger acoustically active particles reach the surface faster than smaller bubbles, and they break and/or deform at the membrane with relatively large pores. In case the frictional forces holding the particles is not sufficient and the particles move with the flow direction the membrane, aided by the ultrasonic field, will prevent them from reentering the main flow stream.

In FIGS. 12G and 12H are schematically shown top and side views of another preferred embodiment of the invention which utilizes the concepts described in connection with the embodiments shown in FIGS. 12A to 12F. In this embodiment the fluid, with the acoustically active particles immersed in it, flows (in the direction of arrows 122) through a tube having a spiral shaped section 131, having entrance 132 and exit 133, and comprising a membrane 125 disposed throughout the length of the spiral section. Transducer 130 emits ultrasound waves in the direction 134 that is orthogonal to the plane of section 131, thus pushing the particles toward the membrane 125. The highest strength of the ultrasonic field is on the central axis of the transducer which is aligned with the center of the spiral section of the tube. As discussed above the smaller the size of bubble the closer it will get to the center before reaching the membrane surface and being neutralized. As the bubbles approach the center of the spiral, they are also approaching the central axis of the transducer and therefore more force is exerted on them. This pushes them with increasing momentum towards the membrane thus neutralizing them more effectively.

Another preferred embodiment of the invention is a method of introducing material encapsulated within acoustically active particles into a vessel through which a fluid is flowing by immersing the particles in the fluid; concentrating the acoustically active particles at a predetermined location within the vascular network; and releasing the encapsulated material at the location, either before or after passing through the vascular membrane into the interstitial fluid, by shrinking and/or breaking apart and/or dissolving the particles.

As an illustrative but nonlimitative example of this embodiment, a description of a method and apparatus for slowing, stopping and accumulating encapsulated drugs at a specific site in the body, for example at the location of a tumor is presented.

Referring to FIG. 11, acoustic source 97 comprised of a single acoustic element or an array of acoustic elements produces a focused ultrasonic field comprised of acoustic pressure waves 95 traveling in the direction indicated by arrows 96. The boundaries of the field are indicated by solid lines 98 and the focus is 94. The acoustic waves are focused (longitudinally and axially) at a designated site (volume) by means well known to skilled persons. The effect of the radiation pressure is greatest in the focal region and decreases in proportion to the distance from the focus, the f number (the relationship between the acoustic source diameter and the distance from it to the focus), the ultrasonic wavelength, and the acoustic properties of the medium.

In the focal zone, the blood flows in different directions inside one or more blood vessels 90, 91, 92 that are not necessarily perpendicular to the direction of propagation of the ultrasound field 96. As a result, depending on the relative angle between the bloodstream and the ultrasonic field propagation, only part of the acoustic radiation force will push bubbles immersed in the bloodstream towards the vessel wall, causing them to stop. In the most extreme case, where the blood flow direction in a vessel in the focal zone is parallel to the direction of wave propagation, the waves will not push the bubble towards the wall but will accelerate it away from the source pushing it towards a vessel wall at the first curve.

A catheter is used to release drugs encapsulated in microbubbles 93 into the artery (or arteries) which bring blood directly to the targeted site. This method of introducing the microbubbles minimizes one of the basic problems of conventional systemic drug delivery methods, i.e. the systemic circulation of the drug until it eventually reaches the targeted site. The artery chosen for the introduction of the microbubbles (91 in FIG. 111) is the one that is as close as possible to perpendicular to the ultrasonic field for the reasons discussed above.

FIGS. 17A to 17C show a non-limiting preferred embodiment of the catheter used to introduce the drugs into the bloodstream. In FIG. 17A, the catheter 151 is shown inserted into the blood vessel 150 using fluoroscopy guidelines or any other insertion technique known in the art. Vessel 150 has two side-branches 157 and 158 and it is desired to introduce the encapsulated drug 154 into branch 157, without allowing any of the drug to enter branch 158 or in any part of vessel 150 beyond branch 157. During insertion of the catheter, the balloon 152 at the tip of the catheter, is deflated allowing free flowing of blood in all branches of the vessel 150 (blood flow direction is indicated by the black arrows). In FIG. 17B is shown the injection method. Before injection (in the direction indicated by double arrow 160) of the encapsulated drug 154 is started, either through a hole in the main tube or a valve 153, the balloon 152 is inflated by gas or a liquid which is delivered to it through side tube 155 or the main tube 156. The inflated balloon diverts all of the blood flow to the specified side-branch 157, thus limiting the systemic spread of the drug. In FIG. 17C, is depicted a situation in which the catheter is deployed against the bloodstream. The balloon and valve can be manufactured in any orientation and distance from each other in order to allow injection of the drug to specific vessel or vessels, also any number of balloons and valves can be used.

Once the microbubbles are introduced into the bloodstream and arrive at the focal zone of the ultrasound field, they are pushed to the wall of the artery, slowed down, stopped, and held in place by the force of the ultrasonic waves as described hereinabove. For this application the minimal acoustic force necessary to accumulate the microbubbles at the targeted site is used at first. Because the drug is encapsulated in very acoustically active microbubbles, by means of ultrasonic imaging, the operator (the physician) can obtain a precise indication of the amount of drugs (number of microbubbles) present at the targeted site. When the operator decides that the uptake process of the encapsulated drugs in the neighborhood of the targeted cells is complete, (numeral 99 in FIG. 11 designates cells that have taken up the encapsulated drug). Special ligands and vectors can be incorporated on the membrane of the microbubbles to allow greater specificity to targeted cells.

A preferred embodiment of the apparatus consists of one or more ultrasound heads with one or more ultrasonic sources (or arrays) to allow focusing energy from several different directions. In other embodiments, in order to allow accurate focusing by the operator, ultrasonic imaging capability can be added, or outside imaging instrument (MRI, C-arm, etc.) can be used in order to accurately find the site to be targeted, and focus the ultrasonic waves on it.

Although embodiments of the invention have been described by way of illustration, it will be understood that the invention may be carried out with many variations, modifications, and adaptations, without departing from its spirit or exceeding the scope of the claims.

1. A method for neutralizing acoustically active particles immersed in a flowing fluid, in which:
(a) ultrasonic waves are provided, which propagate through said fluid, said ultrasonic waves pushing said particles in the direction of a friction layer near a surface or surfaces in contact with said fluid or pushing said particles against the direction of flow of said fluid, thereby causing the motion of said particles to stop; said ultrasonic waves additionally causing said particles to be held in place by pushing them against said surface or surfaces or against the flow of said flowing fluid; and
(b) an acoustic radiation force is provided for neutralizing said ultrasonically active particles which have been stopped and held in place;
characterized in that, said acoustically active particles are neutralized within the flowing fluid by one or a combination of the following processes:
i. a deformation process; wherein an acoustic field is provided which is modulated at a frequency corresponding to a deformation frequency of said acoustically active particles, thereby surrounding said particles with an asymmetric acoustic radiation force and causing fragmentation of said particles into smaller ones; and
ii. a diffusion process; wherein a pulsating acoustic radiation field is provided which alternately compresses and releases said particles, thereby increasing the efficiency of the diffusion of material from the interior of said particle to the surrounding fluid.

2. A method according to claim 1, wherein the acoustic radiation force for pushing and the acoustic radiation force for neutralizing are provided by the same source.

3. A method according to claim 1, wherein the acoustic radiation force for pushing and the acoustic radiation force for neutralizing are provided by different sources.

4. A method according to clam 1, wherein the acoustic radiation force for pushing and the acoustic radiation force for neutralizing are applied as a superimposition of acoustic radiation forces having two or more frequencies and or waveforms.

5. A method according to claim 1, wherein the acoustic radiation force for pushing and the acoustic radiation force for neutralizing have waveforms chosen from the group comprising, but not limited to:
(a) continuous; and
(b) pulsating.

6. A method according to claim 1, wherein the acoustic radiation force for pushing and the acoustic radiation force for neutralizing are aimed towards the surface of a wall of the vessel containing the fluid or a surface placed in their path.

7. A method according to claim 1, wherein the acoustic radiation force for pushing and the acoustic radiation force for neutralizing are aimed in a direction opposite to the direction of flow of the fluid and along the axis of the vessel through which said fluid flows.

8. A method according to claim 1, wherein the acoustic radiation force for pushing and the acoustic radiation force for neutralizing are focused,

9. A method according to claim 1, wherein the acoustic radiation force for pushing and/or the acoustic radiation force for neutralizing are generated upon detection of the acoustically active particles by a detector or detectors.

10. A method according to claim 9, wherein the detector is chosen from the group comprising, but not limited to:
(a) an ultrasonic detector; and
(b) an electro-optic detector.

11. A method according to claim 9, wherein the detection is made by detecting ultrasonic energy sourced emitted by an ultrasonic transducer, refracted by the particles, and detected by said transducer.

12. A method according to claim 9, wherein the detection is made by detecting ultrasonic energy emitted by an ultrasonic transducer, refracted by the particles, and detected by a different transducer.

13. A method according to claim 1, wherein the flow of the fluid is through a vessel that is open to view.

14. A method according to claim 1, wherein the flow of the fluid is through a vessel that is surrounded by an object and therefore is not open to view.

15. A method according to claim 14, wherein the orientation of the vessel is determined with the aid of ultrasonic detectors which detect the flow of fluid through said vessel.

16. A method according to claim 15, wherein the external object is a human body.

17. A method according to claim 16 wherein the vessel is a blood vessel.

18. A method according to claim 16 wherein the vessel is the carotid artery.

19. A method according to claim 1, wherein the surface is one or a plurality of membranes and large acoustically active particles break apart into smaller particles, which pass through the openings in said membranes upon impact.

20. A method according to claim 19, wherein the size of the pores in the membranes is between 0.1 μm to 1 mm.

21. A method according to claim 19 wherein the membranes together with the ultrasonic propagating field acting on the acoustically active particles effectively act as a semi-permeable membrane which permits particles to leave the fluid flow through the pores of said membranes and prevents the particles from reentering the flow by means of: the ultrasonic force alone, as a result of the merger of the smaller bubbles that have passed through the openings in said membrane resulting in a bubble that is larger than said openings, or by a combination of both effects.

22. A method according to claim 19, wherein there is an array of open cells on the side of the membrane surface opposite to the flow of the acoustically active particles and wherein after particles are broken apart and pass through the openings of said membrane, they enter said cells thus preventing them from recombining to form particles whose dimensions exceed that of said cells.

23. A method according to claim 1, wherein the surface comprises an array of cells arranged in a honeycomb pattern.

24. A method according to claim 19, wherein the acoustic pressure exerted on acoustically active particles that are larger than the pore size of the membrane causes them to deform without breaking apart upon impact with said membrane and slip through said pores, regaining their origin shape after slipping through said membrane.

25. A method according to claim 19 where the dimensions of the pores of each succeeding membrane in a plurality of membranes become smaller in the direction of the fluid flow.

26. A method according to claim 1, wherein the acoustically active particles comprise an encapsulated material.

27. A method according to claim 26, wherein the encapsulated material is a drug.

28. An ultrasonic system for using the method of claim 1 to neutralize acoustically active particles immersed in a flowing fluid, said apparatus comprising:
(a) a fluid flow path through a vessel;
(b) a surface which creates a friction layer to the fluid that flows adjacent to it, and can be partially or fully submerged in the fluid, or may consist of a wall of said vessel or a type of membrane;
(c) transducing means acoustically connected to said vessel or submerged in it, said transducing means delivering ultrasonic waves that propagate through said fluid, said ultrasonic waves pushing said particles in the direction of said friction layer near a surface or surfaces in contact with said fluid or pushing said particles against the direction of flow of said fluid;
wherein said transducing means are additionally capable of delivering one or more of the types of acoustic radiation fields selected from the following group:
(i) a pulsating acoustic radiation field, which alternately compresses and releases said particles; and
(ii) an acoustic field which is modulated at a frequency corresponding to a deformation frequency of said acoustically active particles.

29. A system according to claim 28, wherein the surface is a layer of the flowing fluid and the acoustic energy is directed opposite to the direction of flow.

30. A system according to claim 28, wherein the acoustic energy is focused.

31. A system according to claim 29, wherein the fluid flows in a tube.

32. A system according to claim 28, wherein the transducing means comprise an ultrasound head comprising one or more ultrasound transducers.

33. A system according to claim 32, wherein the number of ultrasound transducers is at least three and two of said transducers are used to detect the presence of acoustically active particles and to influence the operation of the remainder of said transducers.

34. A system according to 32, wherein the transducing means are comprised of a disc shaped main transducer surrounded by an outer ring shaped transducer, said outer transducer being driven in an anti-phase manner to said main transducer.

35. A system according to claim 82, wherein the acoustic energy is focused.

36. A system according to claim 32, wherein the acoustic energy is unfocused.

37. A system according to claim 28, wherein the system comprises means for providing ultrasonic energy for selectively stopping, breaking apart, shrinking, and dissolving acoustically active particles immersed in blood flowing in the carotid arteries.

38. A system according to claim 37, further comprising a disposable pillow.

39. A system according to claim 37, wherein the system comprises two ultrasonic heads one located on each carotid artery.

40. A system according to claim 37, comprising two ultrasonic heads each comprising at least two ultrasonic bubble detectors for detect acoustically active particles and/or fluid flow and at least one ultrasonic transducer to provide the ultrasonic energy.

41. A system according to claim 28, wherein the surface is a membrane or has a honeycomb structure to aid in breaking apart and/or holding the acoustically active particles.

42. A system according to claim 41, wherein the membrane acting together with the acoustic energy effectively acts as a semi-permeable membrane, which acts to remove acoustically active particles from the flowing fluid in which they are immersed.

43. A system according to claim 28, wherein the vessel through which the fluid flows is an arterial line of cardiopulmonary machine, contrast media catheter, or dialysis machine or a high-flow venous line.

44. A system according to claim 28, wherein the acoustically active particles comprise encapsulated material.

45. A system according to claim 44, wherein the acoustically active particles are delivered to a selected location in a vessel by the flowing fluid, concentrated at said location within said vessel and the encapsulated material is released at said location by shrinking and/or breaking apart and/or dissolving said particles.

46. A system according to claim 45, wherein the acoustically active particles are introduced into the flowing fluid using a specially designed balloon catheter.

47. A system according to claim 44, wherein the encapsulated material is a drug.

48. A system according to claim 45, wherein the vessel is part of the vascular system of a human or animal body.
